(19) United States
(12) Patent Application Publication
(10) Pub. No.: US 2008/0139908 A1
(43) Pub. Date: Jun. 12, 2008

(54) Multi-Wavelength Spatial Domain Near Infrared Oximeter to Detect Cerebral Hypoxia-Ischemia

(76) Inventor: Charles Dean Kurth, Cincinnati, OH (US)

Correspondence Address: TAFT, STETTINIUS & HOLLISTER LLP, SUITE 1800, 425 WALNUT STREET, CINCINNATI, OH, 45202-3957, US

(21) Appl. No.: 11/939,141

(22) Filed: Nov. 13, 2007

Related U.S. Application Data
(63) Continuation of application No. PCT/US06/18586, filed on May 15, 2006.
(60) Provisional application No. 60/680,752, filed on May 13, 2005.

(51) Int. Cl. A61B 5/1455

(52) U.S. Cl. 600/340

(57) ABSTRACT

Methods and apparatus for measuring cerebral O₂ saturation and detecting cerebral hypoxia-ischemia using multi-wavelength near infrared spectroscopy (NIRS). Near-infrared light produced by an emitter is directed through brain tissue. The intensity of the light that passes through the brain tissue is measured using photodiode detectors positioned at distinct distances from the emitter. This process is conducted for at least three wavelengths of near-infrared light. One of the wavelengths used is substantially at an isobestic point for oxy-hemoglobin and deoxy-hemoglobin, but the other two may be any wavelengths within the near-infrared spectrum (700 nm to 900 nm), so long as one of the additional wavelengths is greater than the isobestic point and the other is less than the isobestic point. Tissue oxygenation is calculated using an algorithm derived from the Beer-Lambert law. Cerebral hypoxia-ischemia may be diagnosed using the calculated tissue oxygenation value.

### CROSS-REFERENCE TO RELATED APPLICATIONS

This application is continuation of PCT Application No. PCT/US2006/018586, filed May 15, 2006, which claims the benefit of provisional Application No. 60/680,752, filed May 13, 2005, the entire disclosures of which are incorporated by reference.

### BACKGROUND OF THE INVENTION

This invention relates to measurement of cerebral O₂ saturation and detection of cerebral hypoxia-ischemia in infants using multi-wavelength near infrared spectroscopy (NIRS).

Despite advances in pediatrics over the years, brain damage from hypoxia-ischemia continues to occur. Populations at high risk of cerebral hypoxia-ischemia include those with congenital heart disease, sickle cell anemia, head trauma, cerebrovascular disease, and critical illnesses such as prematurity and sepsis. The standard method for monitoring for cerebral hypoxia-ischemia is the neurological examination. However, in the above-mentioned populations, this clinical exam is not reliable because of brain immaturity, systemic illness, or use of sedative drugs. Thus, the diagnosis of cerebral hypoxia-ischemia is often delayed for days to weeks, making it impossible to prevent or treat brain damage.

Several technologies exist to monitor the brain, which could be used to detect cerebral hypoxia-ischemia. Electroencephalography and Cerebral Function Monitors (CFM) are robust and non-invasive technologies, although they are insensitive or nonspecific for hypoxia-ischemia in the immature, sedated, or anesthetized brain. Jugular bulb oximetry, magnetic resonance imaging, and positron emission tomography are sensitive to cerebral hypoxia-ischemia; however, they may be undesirable in critically ill infants. Another technology, near-infrared spectroscopy (NIRS), is non-invasive and can be applied at the bedside. It uses near-infrared light (700-900 nm) to monitor oxygenated and deoxygenated hemoglobin in gas exchanging vessels and can detect cerebral hypoxia-ischemia, much like pulse oximetry can detect arterial hypoxia.

### INTRODUCTION TO THE INVENTION

Although NIRS holds promise to detect cerebral hypoxia-ischemia in infants, engineering and indication issues have heretofore hampered its use in clinical practice. Thus far, the NIRS monitors have not been able to provide a number that is clinically relevant, accurate, and/or reliable to diagnose cerebral hypoxia-ischemia. In the past few years, cerebral O₂ saturation has been identified as a number that is clinically relevant, and methods to verify NIRS accuracy have been established. Advances in optical electronics hardware should make it possible to improve the reliability and accuracy of NIRS in the clinical environment. The present invention addresses these concerns and provides a NIRS cerebral oximeter that has hardware features to determine cerebral O₂ saturation in infants.

The present invention is generally directed to methods and apparatuses for measuring cerebral O₂ saturation and detecting cerebral hypoxia-ischemia using multi-wavelength near infrared spectroscopy (NIRS). In practice, an apparatus like the embodiments described herein is secured to the head of a patient believed to be potentially suffering from cerebral hypoxia-ischemia. Then, light of a particular near infrared wavelength is be sent through an amount of brain tissue using a near infrared light emitter positioned on the apparatus. At least three intensities of the light that passes through the amount of brain tissue is then measured using at least three photodiode detectors positioned at distinct distances from the emitter, also located on the apparatus. This process is repeated for at least two other wavelengths of light. One of the wavelengths used is substantially at an isobestic point for oxy-hemoglobin and deoxy-hemoglobin, but the other two may be any wavelengths within the near infrared spectrum (700 nm to 900 nm), so long as one of the additional wavelengths is greater than the isobestic point and the other is less than the isobestic point. To calculate the saturation of tissue oxygenation, the measured intensities are then plugged into an algorithm derived from the Beer-Lambert law and based at least upon one or more ratios of measured intensities at two or more wavelengths and one or more ratios of measured intensities at two or more photodiodes. From the calculated saturation of tissue oxygenation, a physician may determine whether or not the patient suffers from cerebral hypoxia-ischemia.

It is therefore a first aspect of the present invention to provide a method for measuring brain oxygen saturation using multi-wavelength near infrared spectroscopy (NIRS) that includes the steps of: sending light of a first near infrared wavelength through an amount of brain tissue, with the assistance of a near infrared light emitter; measuring a first set of at least three intensities of the light of the first near infrared wavelength that passes through the brain tissue, with the assistance of at least three photodiode detectors positioned at least three distances from the near infrared light emitter; sending light of a second near infrared wavelength through an amount of brain tissue, with the assistance of the near infrared light emitter; measuring a second set of at least three intensities of the light of the second near infrared wavelength that passes through the brain tissue, with the assistance of the at least three photodiode detectors positioned at the at least three distances from the near infrared light emitter; sending light of a third near infrared wavelength through an amount of brain tissue, with the assistance of the near infrared light emitter; measuring at third set of at least three intensities of the light of the third near infrared wavelength that passes, through the brain tissue, with the assistance of at least three photodiode detectors positioned at the at least three distances from the near infrared light emitter; and calculating a saturation of tissue oxygenation using an algorithm derived from the Beer-Lambert law and based at least upon one or more ratios of measured intensities at two or more near infrared wavelengths and one or more ratios of measured intensities at two or more photodiode detectors.

In a further detailed embodiment, the first near infrared wavelength is substantially an isobestic point for oxy-hemoglobin and deoxy-hemoglobin, the second near infrared wavelength is shorter than the first wavelength, and the third near infrared wavelength is longer than the first wavelength. In yet a further detailed embodiment, the method further includes the step of comparing the calculated saturation of tissue oxygenation to one or more sets of medical diagnosis standards to determine an existence of cerebral hypoxia-ischemia or a lack of cerebral hypoxia-ischemia.

It is within the scope of the first aspect of the present invention that the at least three distances include a first distance, a second distance that is incrementally longer than the first distance and a third distance that is incrementally longer than the third distance. And the calculating step includes calculating the saturation of tissue oxygenation, S_{O2}, from substantially the following equation,

$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$

where N_{λ} is the number of wavelength pairs, N_{δ} is the number of emitter-detector distances, R is a ratio of measured intensities at two wavelengths, r is a ratio of measured intensities at detectors, D is the distance between the emitter and detector, and A and B denote lump constants for the extinction coefficients of Hb and HbO₂.

It is a second aspect of the present invention to provide a method for measuring brain oxygen saturation using multi-wavelength near infrared spectroscopy (NIRS) that includes the steps of: (a) sending light of a first near infrared wavelength through an amount of brain tissue, with the assistance of a near infrared light emitter; (b) measuring a first intensity of the light of the first near infrared wavelength that passes through the brain tissue, with the assistance of a first photodiode detector positioned a first distance from the near infrared light emitter; (c) measuring a second intensity of the light of the first near infrared wavelength that passes through the brain tissue, with the assistance of a second photodiode detector positioned a second distance from the near infrared light emitter; (d) measuring a third intensity of the light of the first near infrared wavelength that passes through the brain tissue, with the assistance of a third photodiode detector positioned a third distance from the near infrared light emitter; (e) repeating steps (a) through (d) a plurality of times for light of a corresponding plurality of near infrared wavelengths; and (f) calculating a saturation of tissue oxygenation using an algorithm derived from the Beer-Lambert law and based at least upon one or more ratio of measured intensities at two or more of the near infrared wavelengths and one or more ratios of measured intensities at the two or more photodiode detectors.

In a more detailed embodiment, the first near infrared wavelength is substantially an isobestic point for oxyhemoglobin and deoxyhemoglobin, at least one of the near infrared wavelengths is shorter than the first near infrared wavelength, and at least one of the near infrared wavelengths is longer than the first near infrared wavelength. In a further detailed embodiment the method further includes the step of comparing the calculated saturation of tissue oxygenation to one or more sets of medical diagnosis standards to determine an existence of cerebral hypoxia-ischemia or a lack of cerebral hypoxia-ischemia.

It is within the scope of the second aspect of the present invention that the at least three distances include a first distance, a second distance that is incrementally longer than the first distance and a third distance that is incrementally longer than the third distance. And the calculating step includes calculating the saturation of tissue oxygenation, S_{O2}, from substantially the following equation,

$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$

where N_{λ} is the number of wavelength pairs, N_{δ} is the number of emitter-detector distances, R is a ratio of measured intensities at two wavelengths, r is a ratio of measured intensities at detectors, D is the distance between the emitter and detector, and A and B denote lump constants for the extinction coefficients of Hb and HbO₂.

It is a third aspect of the present invention to provide a method for measuring brain oxygen saturation using multi-wavelength near infrared spectroscopy (NIRS) that includes the steps of: (a) sending light of a first near infrared wavelength through an amount of brain tissue, with the assistance of a near infrared light emitter; (b) measuring a first set of a plurality of intensities of the light of the first near infrared wavelength that passes through the brain tissue, with the assistance of a plurality of photodiode detectors positioned at corresponding plurality distances from the near infrared light emitter; (c) repeating the steps (a) and (b) for light of a plurality of near infrared wavelengths; and (d) calculating a saturation of tissue oxygenation using an algorithm derived from the Beer-Lambert law and based at least upon one or more ratios of measured intensities at two or more of the near infrared wavelengths and one or more ratios of measured intensities at two or more of the photodiode detectors.

In a more detailed embodiment, the first near infrared wavelength is substantially an isobestic point for oxyhemoglobin and deoxyhemoglobin, at least one of the near infrared wavelengths is shorter than the first near infrared wavelength, and at least one of the near infrared wavelengths is longer than the first near infrared wavelength. In yet a further detailed embodiment, the method further includes the step of comparing the calculated saturation of tissue oxygenation to one or more sets of medical diagnosis standards to determine an existence of cerebral hypoxia-ischemia or a lack of cerebral hypoxia-ischemia.

It is within the scope of the third aspect of the present invention that the calculating step includes calculating the saturation of tissue oxygenation, S_{O2}, from substantially the following equation,

$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$

where N_{λ} is the number of wavelength pairs, N_{δ} is the number of emitter-detector distances, R is a ratio of measured intensities at two wavelengths, r is a ratio of measured intensities at detectors, D is the distance between the emitter and detector, and A and B denote lump constants for the extinction coefficients of Hb and HbO₂.

It is a fourth aspect of the present invention to provide a method for measuring brain oxygen saturation using multi-wavelength near infrared spectroscopy (NIRS) that includes the steps of: sending light of a first near infrared wavelength through an amount of brain tissue, with the assistance of a near infrared light emitter; measuring a first intensity of the light of the first near infrared wavelength that passes through the brain tissue, with the assistance of a first photodiode detector positioned a first distance from the near infrared light emitter; measuring a second intensity of the light of the first near infrared wavelength that passes through the brain tissue, with the assistance of a second photodiode detector positioned a second distance from the near infrared light emitter; measuring a third intensity of the light of the first near infrared wavelength that passes through the brain tissue, with the assistance of a third photodiode detector positioned a third distance from the near infrared light emitter; sending a light of a second near infrared wavelength through the amount of brain tissue, with the assistance of the near infrared light emitter; measuring a fourth intensity of light of the second near infrared wavelength that passes through the brain tissue, with the assistance of the first photodiode detector positioned the first distance from the near infrared light emitter; measuring a fifth intensity of light of the second near infrared wavelength that passes through the brain tissue, with the assistance of the second photodiode detector positioned the second distance from the near infrared light emitter; measuring a sixth intensity of light of the second near infrared wavelength that passes through the brain tissue, with the assistance of the third photodiode detector positioned the third distance from the near infrared light emitter; sending a light of a third near infrared wavelength through the amount of brain tissue, with the assistance of the near infrared light emitter; measuring a seventh intensity of light of the third near infrared wavelength that passes through the brain tissue, with the assistance of the first photodiode detector positioned the first distance from the near infrared light emitter; measuring an eighth intensity of light of the third near infrared wavelength that passes through the brain tissue, with the assistance of the second photodiode detector positioned the second distance from the near infrared light emitter; measuring a ninth intensity of light of the third near infrared wavelength that passes through the brain tissue, with the assistance of the third photodiode detector positioned the third distance from the near infrared light emitter; and calculating a saturation of tissue oxygenation using an algorithm derived from the Beer-Lambert law and based at least upon one or more ratios of measured intensities at two or more of the near infrared wavelengths and one or more ratios of measured intensities at two or more of the photodiode detectors.

In a more detailed embodiment, the first near infrared wavelength is substantially an isobestic point for oxy-hemoglobin and deoxy-hemoglobin, the second near infrared wavelength is shorter than the first wavelength, and the third near infrared wavelength is longer than the first wavelength. In yet a further detailed embodiment, the method further includes the step of comparing the calculated saturation of tissue oxygenation to one or more sets of medical diagnosis standards to determine an existence of cerebral hypoxia-ischemia or a lack of cerebral hypoxia-ischemia.

In an alternate detailed embodiment of the fourth aspect of the present invention the first near infrared wavelength is 805 nm, the second near infrared wavelength is 730 nm, the third near infrared wavelength is 850 nm, the first distance is 2 cm, the second distance is 3 cm and the third distance is 4 cm. In a further detailed embodiment, the method further includes the step of comparing the calculated saturation of tissue oxygenation to one or more sets of medical diagnosis standards to determine an existence of cerebral hypoxia-ischemia or a lack of cerebral hypoxia-ischemia.

In yet another alternate detailed embodiment of the fourth aspect of the present invention, the calculating step includes calculating the saturation of tissue oxygenation, S_{O2}, from substantially the following equation,

$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$

where N_{λ} is the number of wavelength pairs, N_{δ} is the number of emitter-detector distances, R is a ratio of measured intensities at two wavelengths, r is a ratio of measured intensities at detectors, D is the distance between the emitter and detector, and A and B denote lump constants for the extinction coefficients of Hb and HbO₂.

It is a fifth aspect of the present invention to provide an apparatus for measuring brain oxygen saturation that includes: a probe housing; a near-infrared light emitter housed within the probe housing; a first photodiode detector positioned a first distance from the emitter; a second photodiode detector positioned at a second distance from the emitter, the second distance being longer than the first distance; and a third photodiode detector positioned at a third distance from the emitter, the third distance being longer than the second distance. In a more detailed embodiment, the probe housing includes an apparatus for securing the housing to the head of a patient.

### BRIEF DESCRIPTION OF THE SEVERAL VIEWS OF THE DRAWINGS

FIG. 1 is an elevation view of an exemplary embodiment of the present invention;

FIG. 2 is a graph showing the response of the wavelength pair intensity ratio (R) for 730 nm and 850 nm measured at the 4 cm detector by the NIRS oximeter, to O₂ saturation (S_{O2}) of blood perfusing the brain model;

FIG. 3 is a graph showing the response of the detector pair intensity ratio (r) for 1 cm and 2 cm detectors measured at 805 nm by the NIRS oximeter, to hemoglobin concentration of blood perfusing the brain model;

FIG. 4 is a graph showing the response of the wavelength pair intensity ratio (R) for 730 nm and 850 nm measured at the 4 cm detector by the NIRS oximeter, to a weighted average of arterial and cerebral venous O₂ saturation (Sm_{O2}) in a piglet hypoxia-ischemia model;

FIG. 5 is a graph showing the NIRS oximeter performance in piglets subjected to hypoxia (n=6) or hypoxia-ischemia (n=7); and

FIG. 6 is a graph showing NIRS oximeter performance in the same piglets used in the test reported in FIG. 4.

### DETAILED DESCRIPTION OF THE INVENTION

The present invention is generally directed to methods and apparatuses for measuring cerebral O₂ saturation and detecting cerebral hypoxia-ischemia using multi-wavelength near infrared spectroscopy (NIRS). In practice, an apparatus like the embodiments described herein is secured to the head of a patient believed to be potentially suffering from cerebral hypoxia-ischemia. An exemplary embodiment of the present invention is designed to be non-invasive—the exemplary apparatus is generally placed such that the bottom surface of the probe is in contact with the outer surface of the patient's forehead, a method requiring no incision or otherwise invasive procedure.

Once the apparatus is secured, light of a particular near infrared wavelength is be sent through an amount of brain tissue using a near infrared light emitter positioned on the apparatus. At least three intensities of the light that passes through the amount of brain tissue is then measured using at least three photodiode detectors positioned at distinct distances from the emitter, also located on the apparatus. This process is repeated for at least two other wavelengths of light. One of the wavelengths used is an isobestic point for oxy-hemoglobin and deoxy-hemoglobin, but the other two may be any wavelengths within the near infrared spectrum (700 nm to 900 nm), so long as one of the additional wavelengths is greater than the isobestic point and the other is less than the isobestic point. To calculate the saturation of tissue oxygenation, the measured intensities are then plugged into an algorithm derived from the Beer-Lambert law and based at least upon one or more ratios of measured intensities at two or more wavelengths and one or more ratios of measured intensities at two or more photodiodes. From the calculated saturation of tissue oxygenation, a physician may determine whether or not the patient suffers from cerebral hypoxia-ischemia.

### 1. ALGORITHM

As with other forms of oximetry, NIRS relies on the Beer-Lambert law, which describes a relationship between light behavior and concentration of a compound:

log(I/Iₒ)=A+S=(ε_{λ}LC)+S (1)

In this expression, I is the measured power of light at the detector after it passes through the tissue, and Iₒ is the measured power of light at the emitter before it enters the tissue. S represents light loss from scattering by the tissue, whereas A represents light loss from absorption by a compound in the tissue. ε_{λ} is the wavelength-dependent molar absorption coefficient of the absorbing compound, L is the path length of the light from emitter to detector, and C is the concentration of the absorbing compound in the tissue.

For the brain, the light absorbing compounds are mainly oxyhemoglobin (HbO₂) and deoxyhemoglobin (Hb), and to a much lesser extent, water and cytochrome aa₃. If multiple compounds that absorb light are present in the tissue, the total absorbance is the sum of the absorbance by all the absorbing compounds. Thus, equation 1 can be transformed to

log(I/Iₒ)=ε^{Hb}L(Hb)+ε^{HbO2}L(HbO₂)+A^{o}+S (2)

where ε^{Hb} and ε^{HbO2} are the molar extinction coefficients of oxy- and deoxy-hemoglobin, and A^{o} is the absorption from other compounds in the tissue (e.g., water, cytochrome aa₃). S_{O2}, an index of tissue oxygenation, is defined in terms of HbO₂ and Hb and Hbₜₒₜₐₗ:

S_{O2}=HbO₂/(Hb+HbO₂)=HbO₂/Hbₜₒₜₐₗ (3)

Hbₜₒₜₐₗ is total hemoglobin concentration per volume of tissue, distinct from blood hemoglobin concentration, which is total hemoglobin concentration per volume of blood. Combining equations 2 and 3 yields

log(I/Iₒ)=ε^{Hb}L(Hbₜₒₜₐₗ−S_{O2}Hbₜₒₜₐₗ)+ε^{HbO2}L(S_{O2}Hbₜₒₜₐₗ)+G (4)

where G represents A^{o} and S, as these terms can be considered one and the same with respect to the measure of I by an instrument. Equation 4 contains unknown variables (L, S_{O2}, Hbₜₒₜₐₗ, and G), measured variables (Iₒ and I), and known constants (ε^{HbO2} and ε^{Hb}) It is possible to solve for S_{O2} through a multi-equation technique in which equations are constructed using the domains of wavelength, time, frequency, or space. However, in our experience, frequency and time domain instrumentation is complex and difficult to reduce to practice. Thus, our approach combines the wavelength and spatial domains.

In the wavelength domain, the emitter and detector distance is held constant and the wavelengths are allowed to vary. For a given wavelength pair at a given emitter and detector distance, assuming constancy of G between the wavelengths, equation 4 can be simplified to

R=aLHbₜₒₜₐₗ+bLHbₜₒₜₐₗS_{O2} (5)

Here, a and b are lump constants for the molar extinction coefficients of Hb and HbO₂ at the 2 wavelengths, and R is the ratio of the measured intensities at the 2 wavelengths. Of note, equation 5 represents a linear function between R and S_{O2} for any given wavelength pair and emitter-detector distance.

In the spatial domain, the wavelength is held constant and the emitter and detector distance is allowed to vary. Path length and emitter-detector distance (D) are related through a constant known as the differential path length factor (F)

L=DF (6)

### For a given detector pair measuring light intensity from a single emitter, assuming constancy of Hb and HbO₂ in the optical field, equation 2 can be developed to yield

r=L₁(ε^{Hb}Hb+ε^{HbO2}HbO₂)−L₂(ε^{Hb}Hb−ε^{HbO2}HbO₂) (7)

where r is the ratio of the measured intensities at the 2 detectors, and L₁ and L₂ are optical path lengths. If the wavelength at which I is measured such that ε^{Hb} and ε^{HbO2} are equal (isobestic point), then equation 7 can be simplified to

r=ε^{Hb}Hbₜₒₜₐₗ(L₁−L₂) (8)

### Combining equations 6 and 8 yields

Hbₜₒₜₐₗ=r/(ε^{Hb}FΔD) (9)

where ΔD represents the distance between the detector pair. Of note, a linear relationship exists between r and Hbₜₒₜₐₗ. Combining the wavelength and spatial domains expressed by equations 5 and 9 and then simplifying gives

R=ArD/ΔD+BrD/ΔDS_{O2} (10)

In which A and B denote lump constants for the extinction coefficients of Hb and HbO₂. Using equation 10, it is possible to construct an algorithm to determine S_{O2} by measuring light intensities at two or more detectors separated by different distances from a light source emitting at two or more wavelengths in which one wavelength is at the isobestic point.

Boundary conditions around the emitter and detector can also affect optical pathlength and light intensity to create error or noise in the measurement of S_{O2} from D, R, and r. (22, 24) Several methods exist to control this effect, including the use light shields around the emitter, detector and tissue; the use of materials and designs to improve coupling between the emitter, detector and tissue; and the application of robust algorithm and signal processing. In our experience, no single method has been satisfactory. Thus, our approach relies on the design of an optical probe housing the emitter and detector to enhance light shielding and light-tissue coupling, as well as algorithm and signal processes to reduce aberrant signals.

For the algorithm, S_{O2} is determined from the average of several wavelength pairs and emitter-detector distances. If equation 10 is solved for S_{O2} in which the detector pair to determine r is 1 cm apart (ΔD=1), then

S_{O2}=(R−rAD)/(rBD) (11)

One skilled in the art would appreciate that similar equations could be derived where the detector pair to determine r is less than or greater than 1 cm apart (ΔD≠1). Expanding equation 11 to include several wavelength pairs and emitter-detector distances yields an expression

$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ} \qquad ( 12 )$$

where N_{λ} is the number of wavelength pairs and N_{δ} is the number of emitter-detector distances. Again, one skilled in the art would appreciate that similar logarithms could be derived using apparatuses in which the detector pair to determine r is greater than or less than 1 cm apart (ΔD≠1).

### 2. TESTING APPARATUS AND PROTOCOLS

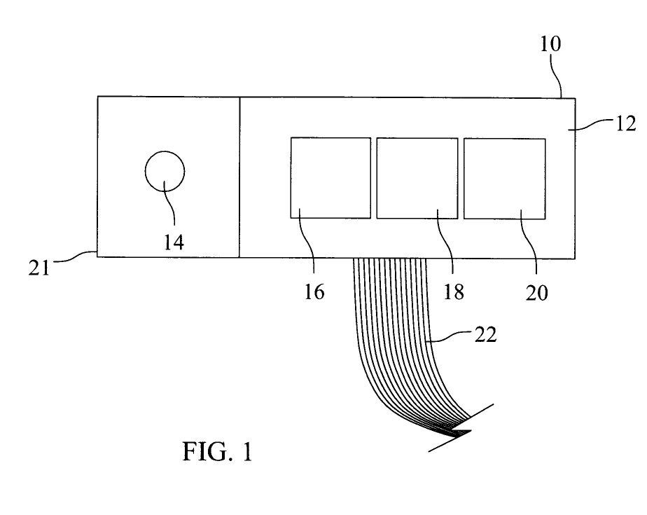

As shown in FIG. 1, a near infrared cerebral oximeter according to an exemplary embodiment of the present invention includes a probe 10 housing a near-infrared light emitter 14 and three photodiode detectors 16, 18 and 20. The exemplary emitter and detectors are recessed (to provide light shielding) within the body of the probe 12, which his made up of a light-weight and highly compliant material to facilitate probe-tissue coupling. The outer surface of the probe is a soft plastic construct 21; and the entire probe is connected to the main unit by a wire bundle 22. The main unit contains the electronic hardware and a computer to capture the signals at each wavelength and detector and to process the captured signals as described herein.

The exemplary emitter 14 contains light emitting diodes at 730 nm, 805 nm, and 850 nm. The 805 nm wavelength is the only known isobestic point for oxy- and deoxy-hemoglobin within the near infrared spectrum. Thus, it is desired that the emitter used contain a light emitting diode at or substantially at the isobestic point of 805 nm. One skilled in the art in the art would recognize though, that any two (or more) additional wavelength could be chosen within the infrared spectrum, so long as one additional wavelength is shorter than 805 nm and one additional wavelength is longer than 805 nm. Testing has shown, in fact, that the farther each addition wavelength is from the isobestic point (and thus the greater the difference in extinction coefficients for Hb and HbO₂ for each additional wavelength), the better the results obtained. Additionally, one skilled in the art would appreciate that more than three wavelengths could be used, provided the emitter used contained additional diodes.

The three photodiode detectors 16, 18 and 20 of the exemplary embodiment may be separated from the emitter 14 at distances of 2 cm, 3 cm and 4 cm, respectively. These distances were selected with regard for the skull size of the infants with which the exemplary embodiment was intended for use. One skilled in the art would appreciate that the desirable emitter-detector distances could vary depending upon the size of the patient, or the nature of the probe. Testing has shown that the larger the emitter-detector distances, the better the results. Therefore, the largest practicable distances are likely the most desirable. For example, a probe to be used with adult patients may facilitate greater emitter-detector distances. Additionally, an alternative exemplary embodiment could be constructed whereby the detectors are be placed on fiber optic lines and inserted into or among the brain tissue. The distances in such a case would likely be mere millimeters or less. In addition to patient size and probe characteristics, technological limitations may also limit the distances used. The largest practicable distance therefore could also be dependent upon the particular optical power of the emitter used or emitter side effects, including the amount of heat generated by the emitter.

During developmental studies of the apparatus and methods of the present invention, this hardware was used to conduct a testing program including two testing models: a “static” brain model and a model using live piglets.

An in-vitro “static” brain model consisting of India ink and intralipid admixed in a gelatin polymer to mimic the optical density of the human head was used to determine instrument signal-to-noise and drift. An in-vitro “dynamic” brain model simulating the brain was used to test equations 5, 6, and 9 and to develop the algorithm. This model consists of a solid plastic structure containing a microvascular network perfused with human blood in which S_{O2} and Hbₜₒₜₐₗ could be varied. Blood S_{O2} was measured by CO-Oximetry (OSM™ 3, Hemoximeter™, Radiometer Copenhagen, Copenhagen NV, Denmark). Blood gas analysis were performed by iSTAT Corporation, Princeton, N.J., USA. pH and PCO₂ were maintained at 7.35 to 7.45 and 35-45 torr, respectively.

A piglet model was used to test equations 5 and 6, to develop the algorithm, and test the algorithm prospectively. After approval by the Institutional Animal Care and Use Committee, 13 piglets aged 4-6 days were studied. Anesthesia was induced with intramuscular ketamine (33 mg/kg) and acepromazine (3.3 mg/kg) and maintained with fentanyl (25 μg/kg bolus, then 10 μg/kg/hr) and midazolam (0.2 mg/kg bolus, then 0.1 mg/kg/hr). Following tracheal intubation and mechanical ventilation, catheters were inserted into the femoral artery and superior sagittal sinus to sample blood for arterial saturation (Sa_{O2}) and cerebral venous saturation (Ss_{O2}). In six piglets, an incision was made in the neck and the carotid arteries were isolated and ligatures were placed around the vessels to produce cerebral ischemia.

In the static model, 3 hours of NIRS data were recorded to determine “static” signal-to-noise and signal drift. To determine the effect of probe positioning on signal-to-noise (“dynamic” signal-to-noise), the probe was repositioned 15 times in the same location. NIRS data was recorded before and after each positioning. In the dynamic model, equation 5 was examined by increasing SO₂ from 0% to 100% and blood samples from the model and NIRS data were recorded at each increment. Experiments were performed at two different blood Hbₜₒₜₐₗ. To test equation 9, Hbₜₒₜₐₗ was decreased from 15 g/dl to 5 g/dl in 2 g/dl steps while S_{O2} was held at 70%. Blood samples from the model and NIRS data were recorded at each hemoglobin concentration.

In the piglet experiments, inspired O₂ (FiO₂), minute ventilation, and cerebral perfusion were varied to force cerebral O₂ saturation over a wide range during high and low cerebral blood volume conditions. Piglets were divided into a hypoxia group and a hypoxia-ischemia group, in which the hypoxia-ischemia group had the carotid arteries occluded during the hypoxia conditions. The following conditions were produced. 1) Normoxia: room air was inspired with minute ventilation adjusted to normocapnia. 2) Hypercapnia/Hyperoxia: FiO₂ was 100% and minute ventilation was decreased to a 10% expired CO₂. 3) Mild Hypoxia: FiO₂ was decreased to 17%. 4) Moderate Hypoxia FiO₂ was decreased to 13%. 5) Severe Hypoxia: FiO₂ was decreased to 8%. After 5 minutes at the condition, NIRS SO₂ (Sc_{O2}), arterial pressure, arterial blood gases and pH, Sa_{O2} and Ss_{O2} were recorded.

### 3. DATA ANALYSIS

Data are presented as mean ±SD. For the static model, drift was

Drift=100*[(Initial Sc_{O2}−End Sc_{O2})/Initial Sc_{O2}]/3 hours (11)

where the initial and end Sc_{O2} represent the average Sc_{O2} over five minutes at the beginning and end of the 3 hour experiment. Instrument “static” signal to noise and “dynamic” signal to noise were

Signal to noise=100*(SD of Sc_{O2})/(average Sc_{O2}) (12)

where the average Sc_{O2} is the average value over 3 hours for the “static” signal to noise, and the average of the 15 values from positioning for the “dynamic” signal to noise.

For the dynamic model, linearity of R and S_{O2} (equation 5), and r and Hbₜₒₜₐₗ (equation 9), were determined by Least Squares Regression. For the piglet model, blood S_{O2} in the cerebrovasculature may be approximated by Sm_{O2}, a weighted average of Sa_{O2} and Ss_{O2}:

Sm_{O2}=0.85(Sa_{O2})+0.15(Ss_{O2}) (13)

The linearity of R and Sm_{O2} (equation 5) was examined in one piglet by Least Squares Regression. The algorithm (equation 10) was then developed from the dynamic in-vitro and piglet models. The accuracy of this algorithm was evaluated in terms of brain oxygen saturation (Sb_{O2}) by Least Squared Regression, as well as by the bias and precision, where Sb_{O2} is defined as the average of Sm_{O2} and Sc_{O2}. Unpaired T-tests with bonferroni correction was used to compare Sc_{O2}, Sa_{O2}, and Ss_{O2} between conditions.

### 4. RESULTS

The following tables and the referenced figures summarize the results of the exemplary NIRS device performance in the static brain and piglet models.

TABLE 1
NIRS cerebral oximeter performance in a static brain model.

| | 2 | | | 3 | | | 4 | | | |
|---|---|---|---|---|---|---|---|---|---|---|
| E-D (cm) | A | B | C | A | B | C | A | B | C | Mean |
| Drift (%/hr) | −1.0 | −1.2 | 2.1 | −1.8 | −1.7 | −0.2 | −0.9 | −1.4 | 0.3 | −0.6 |
| Static s/n (%) | 0.1 | 0.3 | 1.1 | 0.3 | 0.2 | 1.9 | 0.2 | 0.3 | 0.2 | 0.6 |
| Dynamic s/n (%) | 12 | 8.0 | 9.0 | 9.0 | 9.0 | −9.0 | 1.0 | 1.0 | −1.0 | 2.6 |

A, B, and C represent wavelength intensity ratios for 730 nm/850 nm, 730 nm/805 nm and 850 nm/805 nm, respectively, s/n is signal to noise. E-D is the distance between emitter and detector.

Table 1 displays exemplary NIRS device performance in the static brain model. Drift varied from 2%/hr to −2%/hr among the ratios and detectors, the average being <1%. “Static” signal to noise ranged from 0.1% to 1.9% among the ratios and detectors, the average being <1%. The “dynamic” signal to noise ranged from 12% to −9% among the ratios and detectors, the average being 2.6%. The “dynamic” signal to noise was significantly less at the 4 cm emitter-detector than at the 2 and 3 cm emitter-detectors (p<0.001). The “dynamic” signal to noise was significantly greater than the “static” signal to noise at each emitter-detector (p<0.001).

TABLE 2
NIRS oximeter performance in a dynamic brain model.

| E-D (cm) | 2 | | | 3 | | | 4 | | |
|---|---|---|---|---|---|---|---|---|---|
| Ratio vs 8_{O2} | A | B | C | A | B | C | A | B | C |
| Slope | 38 | 31 | −18 | 51 | 40 | −24 | 62 | 51 | −33 |
| intercept | 37 | 47 | 121 | 35 | 46 | 122 | 19 | 29 | 129 |
| r² | 0.99 | 0.99 | 0.99 | 0.98 | 0.99 | 0.99 | 0.98 | 0.98 | 0.98 |

Abbreviations same as Table 1. Slope, intercept, and r² are for the line between the wavelength pair intensity ratio (Ratio) calculated by the device and O₂ saturation (SO₂) of blood perfusing the brain model.

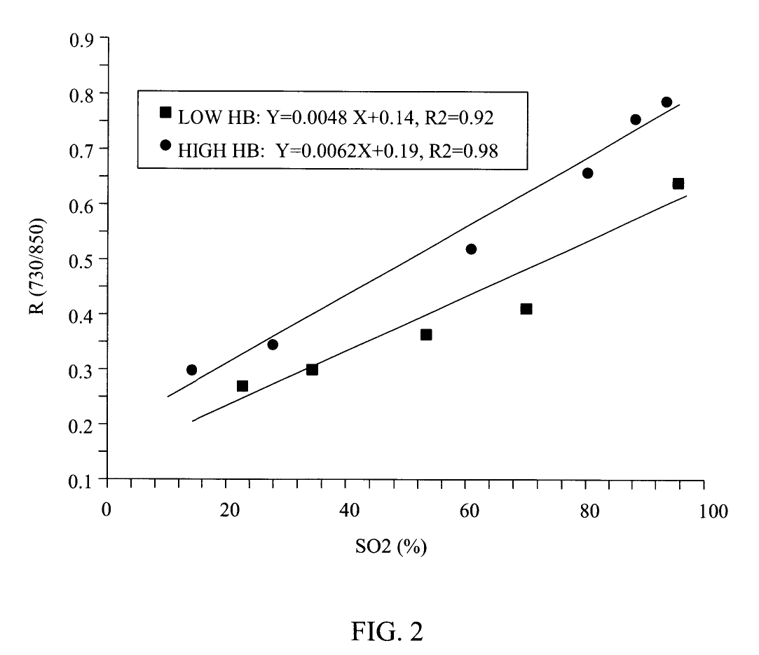

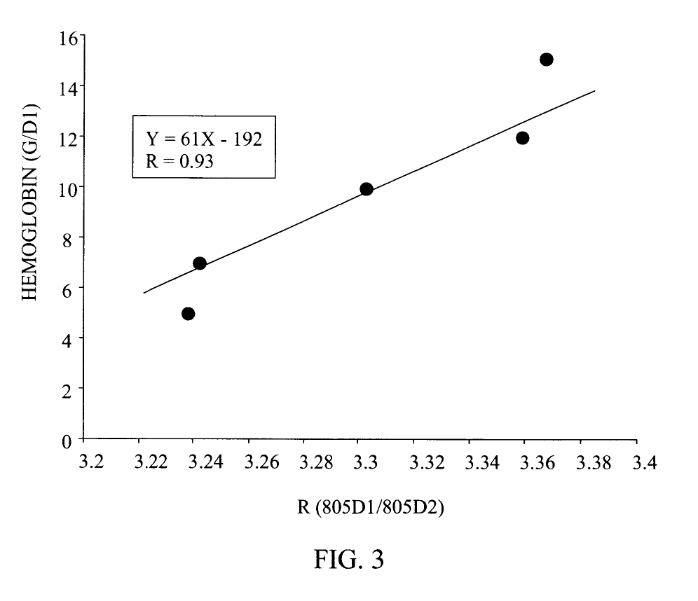

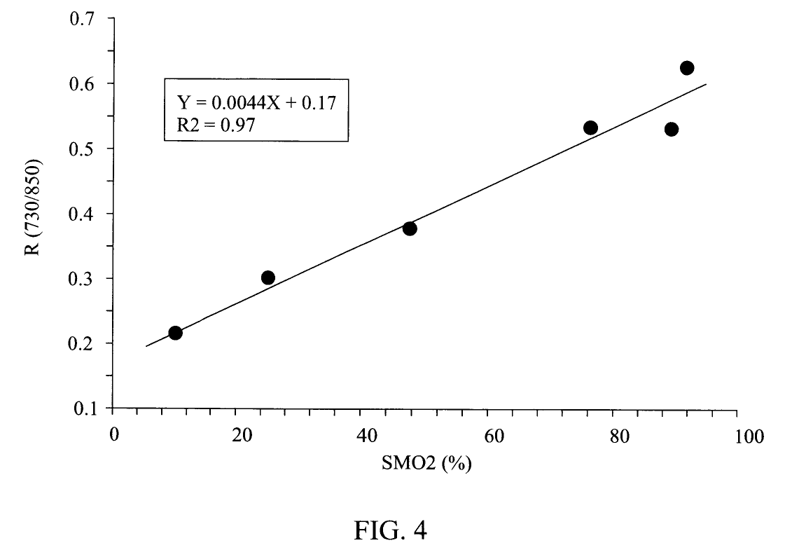

Table 2 and FIGS. 3 and 4 illustrate the dynamic brain model experiments. Linear relationships with excellent correlations were observed between S_{O2} and the intensity ratios for all wavelength pairs (R) at all detectors (table 2), verifying the relationship in equation 5. Slopes and intercepts increased significantly as emitter-detector distance increased (all p<0.01), in keeping with the effect of the longer path length at the greater emitter-detector distances in equations 5 and 6. Although linear relationships were observed at each hemoglobin concentration (FIG. 2), the slopes and intercepts were significantly greater at the higher hemoglobin concentration compared with those at the lower hemoglobin concentration, as predicted in equation 5. A linear relationship was observed between hemoglobin concentration and intensity ratios at 2 detectors (r) for 805 nm (isobestic wavelength for Hb and HbO₂), as predicted from equation 9.

TABLE 3
NIRS oximeter performance in a piglet hypoxia-ischemia model

| E-D (cm) | 2 | | | 3 | | | 4 | | |
|---|---|---|---|---|---|---|---|---|---|
| Ratio vs Sm_{O2} | A | B | C | A | B | C | A | B | C |
| Slope | 27 | 22 | −12 | 34 | 26 | −18 | 44 | 36 | −28 |
| intercept | 34 | 38 | 109 | 35 | 42 | 114 | 17 | 23 | 119 |
| r² | 0.97 | 0.96 | 0.94 | 0.98 | 0.98 | 0.92 | 0.97 | 0.96 | 0.92 |

Abbreviations are as Table 1. Slope, intercept, and r² are for the line between the wavelength pair intensity ratios (Ratio) calculated by the device and weighted average of arterial and cerebral venous blood (Sm_{O2})

Table 3 and FIG. 4 display the results from the piglet experiment examining the relationship between Sm_{O2} and the intensity ratios for the various wavelength pairs. Linear relationships with very good correlation coefficients existed for all ratios at all emitter-detector distances, verifying equation 5 in an in-vivo model. The slopes and intercepts were different than those in the in-vitro brain model, reflecting differences in Hbₜₒₜₐₗ and/or path length between the piglet and model.

TABLE 4
Physiological parameters in the piglet hypoxia-ischemia model.

| | | PCO₂ | PO₂ | MAP |
|---|---|---|---|---|
| Condition | pH | (torr) | (torr) | (mmHg) |
| Normoxia | 7.47 ± 0.06 | 36 ± 4 | 77 ± 8 | 86 ± 13 |
| Hypercapnic- | 7.13 ± 0.05 | 90 ± 14 | 401 ± 84 | 83 ± 13 |
| hyperoxia | | | | |
| Hypoxia 17% | 7.52 ± 0.09 | 33 ± 3 | 59 ± 9 | 83 ± 9 |
| Hypoxia 13% | 7.49 ± 0.08 | 34 ± 3 | 36 ± 10 | 80 ± 12 |
| Hypoxia 9% | 7.48 ± 0.01 | 35 ± 3 | 22 ± 4 | 68 ± 13 |

Values are mean ± SD, n = 13. PCO₂, PO₂, and MAP represent arterial partial pressure of carbon dioxide and oxygen, and mean arterial pressure, respectively.

TABLE 5
Arterial, cerebral venous sinus, and NIRS cerebral
O₂ saturation in the piglet model.

| | Sa_{O2} (%) | | Ss_{O2} (%) | | Sc_{O2} (%) | |
|---|---|---|---|---|---|---|
| Condition | H | H-I | H | H-I | H | H-I |
| Normoxia | 96 ± 1 | 96 ± 3 | 40 ± 3 | 44 ± 9 | 55 ± 7 | 54 ± 5 |
| Hypercapnic- | 99 ± 3 | 100 ± 0 | 88 ± 6 | 87 ± 7 | 93 ± 10 | 89 ± 8 |
| hyperoxia | | | | | | |
| Hypoxia 17% | 93 ± 4 | 90 ± 4 | 35 ± 11 | 19 ± 6* | 46 ± 10 | 38 ± 8 |
| Hypoxia 13% | 75 ± 15 | 64 ± 13 | 23 ± 15 | 14 ± 3 | 25 ± 9 | 24 ± 6 |
| Hypoxia 9% | 46 ± 15 | 36 ± 14 | 14 ± 5 | 6 ± 1* | 17 ± 9 | 12 ± 2 |

Values are mean ± SD. N = 7 for H and n = 6 for HI. H is hypoxia only; H-I is hypoxia and ischemia. *p < 0.01 H vs. HI. Sa_{O2}, Ss_{O2}, and Sc_{O2} are arterial, sagittal sinus (cerebral venous), and NIRS cerebral O₂ saturation, respectively.

Tables 4 and 5 list the blood-gases and pH, as well as Sa_{O2}, Ss_{O2}, and NIRS Sc_{O2} in the test piglet experiments. Arterial blood values were not significantly different between the hypoxia and hypoxia-ischemia groups, except at 17% and 9% FiO₂, where Ss_{O2} in the hypoxia group was significantly greater than in the hypoxia-ischemia group.

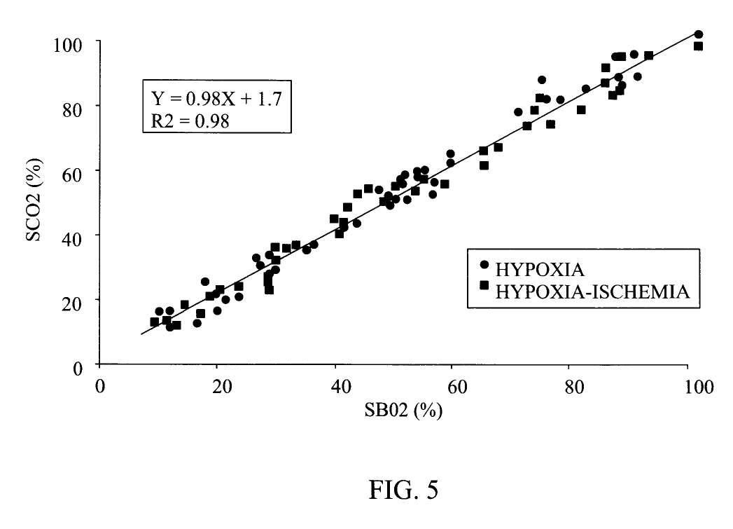

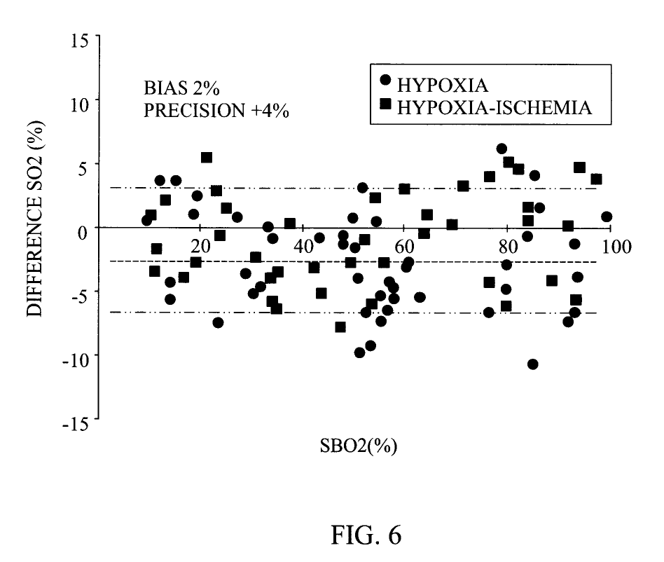

FIGS. 6 and 7 display the device algorithm performance during the test piglet experiments. There was a linear relationship between the NIRS Sc_{O2} and Sb_{O2} with excellent correlation (FIG. 5). The bias and precision for the NIRS cerebral oximeter measured Sc_{O2} was 2% and 4%, respectively, relative to Sb_{O2} (FIG. 6). Instrument performance was similar in both the hypoxia and hypoxia-ischemia groups.

### 5. CONCLUSIONS

Hypoxia-ischemic brain injury and poor neurological outcome continue to occur in certain pediatric populations. Detection and reversal of cerebral hypoxia-ischemia remains the best strategy to improve neurological outcome, as opposed to treating brain injury after it has occurred. The present study developed a NIRS spatial domain construct to detect cerebral hypoxia-ischemia, built a device to use the construct, evaluated the device and construct in several brain models, and tested an algorithm using the construct in a piglet hypoxia-ischemia model. The results demonstrated reliable performance of the device, verified key equations in the construct, and observed accurate measurement of cerebral O₂ saturation.

Cerebral oxygenation can be measured in terms of oxyhemoglobin concentration (Hb_{O2}), oxy-deoxy hemoglobin concentration difference (Hb_{diff}), O₂ saturation (Sc_{O2} or rS_{O2}), and tissue oxygenation index (Ti_{O2}). Because HbO₂ and Hb_{diff} are mass per volume of tissue measurements, they indicate tissue oxygenation. NIRS O₂ saturation and oxygenation index are ratios of oxyhemoglobin concentration to total hemoglobin concentration in the tissue. Because O₂ flux from blood to the mitochondria is driven by O₂ partial pressure linked to the oxy-hemoglobin dissociation curve, Sc_{O2} and Ti_{O2} also indicate tissue oxygenation. For historical reasons, clinicians use O₂ saturation to describe blood oxygenation. We selected O₂ saturation as the term to describe cerebral oxygenation because it is a clinically user-friendly term and other instruments exist to measure it (e.g. CO-Oximetry), which helps with validation.

Experimental methods to validate NIRS cerebral O₂ saturation have heretofore been problematic. Validation requires a comparison of the measurements made by the new device against a standard; the accuracy of the new device is then expressed in terms of bias and precision relative to the standard. In the validation of pulse-oximetry, arterial O₂ saturation measured by pulse-oximetry was compared with that measured by CO-oximetry, the standard. NIRS monitors a mixed vascular bed dominated by gas-exchanging vessels, especially venules. Because no other device measures such a mixed vascular oxygenation, NIRS has lacked a standard to validate it. However, in situations where no standard exists, it is still possible to validate a device using a surrogate which approximates the standard: the surrogate is typically the average of two or more values that should be close to the standard. Accordingly, we used Sb_{O2} as a surrogate, it being the average of Sm_{O2} and Sc_{O2}. Sm_{O2} is close to cerebral O₂ saturation. Sc_{O2} should also be close to cerebral O₂ saturation, given the validation of key equations in the construct, which occurred through the use of our in-vitro model. This model simulates the brain microvasculature monitored by NIRS, and used CO-Oximetry, a standard to measure O₂ saturation.

To determine O₂ saturation by NIRS, it is preferable to account for the effects of light scattering and optical path length. Time-domain, frequency-domain, or spatial domain principles were developed for this purpose. We used the spatial domain because of advantages in engineering and costs over time- and frequency domain technologies. The spatial domain requires equality of light scattering among the wavelengths and equality of O₂ saturation in the optical fields among the emitter-detectors (see equations 5 and 8). The results indicate that these assumptions were reasonably met for the models employed in our study.

The limitation of spatial-domain relates to the different optical paths among the emitter-detector combinations. Computer simulations have shown three possible routes for photons to take after leaving the emitter: 1) shallow—these photons deflect off the scalp, skull, or neocortical surface and never make it to the detectors; 2) deep—these photons scatter indefinitely and never exit from the head; and 3) middle—these photons encounter multiple scattering events before making their way to the detectors. The “middle” photons appear to travel within a banana shaped field, the depth of which is approximately one third the emitter-detector distance. Our optical probe contained emitter-detector distances of 2, 3, and 4 cm, which would give penetration depths of approximately 0.6 cm, 1 cm, and 1.3 cm, corresponding to the “bananas” being in the neocortex for the 2 cm emitter-detector, and neocortex and basal ganglia for the 3 cm and 4 cm emitter-detector.

In light of the various optical paths and tissue regions being monitored, we identified the following errors that could occur with our device. First, it might not detect focal ischemia outside the optical field. For instance, a probe located on the forehead and monitoring the frontal neocortex and basal ganglia would not detect ischemia in the occipital neocortex or deep in the thalamus. Second, it might not detect highly focal ischemia within an optical field that was otherwise well oxygenated. For example, the probe would not detect a lacunar infarct or laminar ischemia in white matter while the overlying grey matter was normally oxygenated. Thus, the spatial domain device is best suited to detect global cerebral hypoxia-ischemia, as in our piglet model.

Motion artifact and other noise have heretofore posed challenges for clinical use of NIRS and other optical devices. In our study, the dynamic signal to noise at the individual detectors was up to 12% and of a magnitude that could be troublesome in the clinical environment. Cerebral O₂ saturation in healthy humans and piglets is 55-80%. After cerebral O₂ saturation decreases to less than 45%, brain function becomes disturbed. Thus, the cushion between normal and dysfunctional can be as low as 10% and within the range of dynamic noise at individual detectors. This dynamic noise would manifest clinically during movement of the probe on the head (motion artifact) or re-positioning the probe on the head. We employed several solutions to minimize this noise. First, the use of wavelength ratios in the algorithm helped because both wavelengths should experience the same noise. Second, the use of multiple ratios in the algorithm helped more, because the noise at the various detectors was positive and negative. Third, the use of longer emitter-detector distances on the probe reduced noise (see table 1). Fourth, the optical probe design permitted its attachment to the head with fewer forces tending to unseat it.

In summary, our exemplary NIRS device uses inexpensive, robust, and clinically friendly technology similar to pulse-oximetry. The device uses a multi-wavelength spatial domain construct and is sufficiently accurate to diagnose cerebral hypoxia-ischemia.

While exemplary embodiments of the invention have been set forth above for the purpose of disclosure, modifications of the disclosed embodiments of the invention as well as other embodiments thereof may occur to those skilled in the art. Accordingly, it is to be understood that the inventions contained herein are not limited to the above precise embodiments and that changes may be made without departing from the scope of the invention as defined by the claims. Likewise, it is to be understood that the invention is defined by the claims and it is not necessary to meet any or all of the stated advantages or objects of the invention disclosed herein to fall within the scope of the claims, since inherent and/or unforeseen advantages of the present invention may exist even though they may not have been explicitly discussed herein.

What is claimed is:

1. A method for measuring brain oxygen saturation using multi-wavelength near infrared spectroscopy (NIRS), comprising the steps of:
sending light of a first near infrared wavelength through an amount of brain tissue, with the assistance of a near infrared light emitter;
measuring a first set of at least three intensities of the light of the first near infrared wavelength that passes through the brain tissue, with the assistance of at least three photodiode detectors positioned at least three distances from the near infrared light emitter;
sending light of a second near infrared wavelength through an amount of brain tissue, with the assistance of the near infrared light emitter;
measuring a second set of at least three intensities of the light of the second near infrared wavelength that passes through the brain tissue, with the assistance of the at least three photodiode detectors positioned at the at least three distances from the near infrared light emitter;
sending light of a third near infrared wavelength through an amount of brain tissue, with the assistance of the near infrared light emitter;
measuring a third set of at least three intensities of the light of the third near infrared wavelength that passes through the brain tissue, with the assistance of at least three photodiode detectors positioned at the at least three distances from the near infrared light emitter; and
calculating a saturation of tissue oxygenation using an algorithm derived from the Beer-Lambert law and based at least upon one or more ratios of measured intensities at two or more near infrared wavelengths and one or more ratios of measured intensities at two or more photodiode detectors.

2. The method of claim 1, wherein the first near infrared wavelength is substantially an isobestic point for oxy-hemoglobin and deoxy-hemoglobin, the second near infrared wavelength is shorter than the first wavelength, and the third near infrared wavelength is longer than the first wavelength.

3. The method of claim 2, further comprising the step of:
comparing the calculated saturation of tissue oxygenation to one or more sets of medical diagnosis standards to determine an existence of cerebral hypoxia-ischemia or a lack of cerebral hypoxia-ischemia.

4. The method of claim 2, wherein the at least three distances include a first distance, a second distance that is incrementally longer than the first distance and a third distance that is incrementally longer than the third distance.

5. The method of claim 4, wherein the calculating step includes calculating the saturation of tissue oxygenation, S_{O2}, from substantially the following equation,
$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$
where N_{λ} is a number of wavelength pairs, N_{δ} is a number of emitter-detector distances, R is a ratio of measured intensities at two wavelengths, r is a ratio of measured intensities at detectors, D is a distance between the emitter and detector, and A and B are lump constants for extinction coefficients of Hb and HbO₂.

6. A method for measuring brain oxygen saturation using multi-wavelength near infrared spectroscopy (NIRS), comprising the steps of:
(a) sending light of a first near infrared wavelength through an amount of brain tissue, with the assistance of a near infrared light emitter;
(b) measuring a first intensity of the light of the first near infrared wavelength that passes through the brain tissue, with the assistance of a first photodiode detector positioned a first distance from the near infrared light emitter;
(c) measuring a second intensity of the light of the first near infrared wavelength that passes through the brain tissue, with the assistance of a second photodiode detector positioned a second distance from the near infrared light emitter;
(d) measuring a third intensity of the light of the first near infrared wavelength that passes through the brain tissue, with the assistance of a third photodiode detector positioned a third distance from the near infrared light emitter;
(e) repeating steps (a) through (d) a plurality of times for light of a corresponding plurality of near infrared wavelengths; and
(f) calculating a saturation of tissue oxygenation using an algorithm derived from the Beer-Lambert law and based at least upon one or more ratio of measured intensities at two or more of the near infrared wavelengths and one or more ratios of measured intensities at the two or more photodiode detectors.

7. The method of claim 6, wherein the first near infrared wavelength is substantially an isobestic point for oxyhemoglobin and deoxyhemoglobin, at least one of the near infrared wavelengths is shorter than the first near infrared wavelength, and at least one of the near infrared wavelengths is longer than the first near infrared wavelength.

8. The method of claim 7, further comprising the step of:
comparing the calculated saturation of tissue oxygenation to one or more sets of medical diagnosis standards to determine an existence of cerebral hypoxia-ischemia or a lack of cerebral hypoxia-ischemia.

9. A method for measuring brain oxygen saturation using multi-wavelength near infrared spectroscopy (NIRS), comprising the steps of:
(a) sending light of a first near infrared wavelength through an amount of brain tissue, with the assistance of a near infrared light emitter;
(b) measuring a first set of a plurality of intensities of the light of the first near infrared wavelength that passes through the brain tissue, with the assistance of a plurality of photodiode detectors positioned at corresponding plurality distances from the near infrared light emitter;
(c) repeating the steps (a) and (b) for light of a plurality of near infrared wavelengths; and
(d) calculating a saturation of tissue oxygenation using an algorithm derived from the Beer-Lambert law and based at least upon one or more ratios of measured intensities at two or more of the near infrared wavelengths and one or more ratios of measured intensities at two or more of the photodiode detectors.

10. The method of claim 9, wherein the first near infrared wavelength is substantially an isobestic point for oxyhemoglobin and deoxyhemoglobin, at least one of the near infrared wavelengths is shorter than the first near infrared wavelength, and at least one of the near infrared wavelengths is longer than the first near infrared wavelength.

11. The method of claim 10, further comprising the step of:
comparing the calculated saturation of tissue oxygenation to one or more sets of medical diagnosis standards to determine an existence of cerebral hypoxia-ischemia or a lack of cerebral hypoxia-ischemia.

12. The method of claim 10, wherein the calculating step includes calculating the saturation of tissue oxygenation, S_{O2}, from substantially the following equation,
$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$
where N_{λ} is a number of wavelength pairs, N_{δ} is a number of emitter-detector distances, R is a ratio of measured intensities at two wavelengths, r is a ratio of measured intensities at detectors, D is a distance between the emitter and detector, and A and B are lump constants for extinction coefficients of Hb and HbO₂.

13. A method for measuring brain oxygen saturation using multi-wavelength near infrared spectroscopy (NIRS), comprising the steps of:
sending light of a first near infrared wavelength through an amount of brain tissue, with the assistance of a near infrared light emitter;
measuring a first intensity of the light of the first near infrared wavelength that passes through the brain tissue, with the assistance of a first photodiode detector positioned a first distance from the near infrared light emitter;
measuring a second intensity of the light of the first near infrared wavelength that passes through the brain tissue, with the assistance of a second photodiode detector positioned a second distance from the near infrared light emitter;
measuring a third intensity of the light of the first near infrared wavelength that passes through the brain tissue, with the assistance of a third photodiode detector positioned a third distance from the near infrared light emitter;
sending a light of a second near infrared wavelength through the amount of brain tissue, with the assistance of the near infrared light emitter;
measuring a fourth intensity of light of the second near infrared wavelength that passes through the brain tissue, with the assistance of the first photodiode detector positioned the first distance from the near infrared light emitter;
measuring a fifth intensity of light of the second near infrared wavelength that passes through the brain tissue, with the assistance of the second photodiode detector positioned the second distance from the near infrared light emitter;
measuring a sixth intensity of light of the second near infrared wavelength that passes through the brain tissue, with the assistance of the third photodiode detector positioned the third distance from the near infrared light emitter;
sending a light of a third near infrared wavelength through the amount of brain tissue, with the assistance of the near infrared light emitter;
measuring a seventh intensity of light of the third near infrared wavelength that passes through the brain tissue, with the assistance of the first photodiode detector positioned the first distance from the near infrared light emitter;
measuring an eighth intensity of light of the third near infrared wavelength that passes through the brain tissue, with the assistance of the second photodiode detector positioned the second distance from the near infrared light emitter;
measuring a ninth intensity of light of the third near infrared wavelength that passes through the brain tissue, with the assistance of the third photodiode detector positioned the third distance from the near infrared light emitter; and
calculating a saturation of tissue oxygenation using an algorithm derived from the Beer-Lambert law and based at least upon one or more ratios of measured intensities at two or more of the near infrared wavelengths and one or more ratios of measured intensities at two or more of the photodiode detectors.

14. The method of claim 13, wherein the first near infrared wavelength is substantially an isobestic point for oxy-hemoglobin and deoxy-hemoglobin, the second near infrared wavelength is shorter than the first wavelength, and the third near infrared wavelength is longer than the first wavelength.

15. The method of claim 14, further comprising the step of:
comparing the calculated saturation of tissue oxygenation to one or more sets of medical diagnosis standards to determine an existence of cerebral hypoxia-ischemia or a lack of cerebral hypoxia-ischemia.

16. The method of claim 13, wherein the first near infrared wavelength is 805 nm, the second near infrared wavelength is 730 nm, the third near infrared wavelength is 850 nm, the first distance is 2 cm, the second distance is 3 cm and the third distance is 4 cm.

17. The method of claim 16, further comprising the step of:
comparing the calculated saturation of tissue oxygenation to one or more sets of medical diagnosis standards to determine an existence of cerebral hypoxia-ischemia or a lack of cerebral hypoxia-ischemia.

18. The method of claim 14, wherein the calculating step includes calculating the saturation of tissue oxygenation, S_{O2}, from substantially the following equation,
$${S}_{O 2} = [ {\sum }_{i = 1}^{{N}_{λ}} {\sum }_{j = 1}^{{N}_{δ}} ( {R}_{i , j} - {r}_{j} {A}_{i} {D}_{j} ) / ( {r}_{j} {B}_{i} {D}_{j} ) ] / {N}_{λ} {N}_{δ}$$
where N_{λ} is a number of wavelength pairs, N_{δ} is a number of emitter-detector distances, R is a ratio of measured intensities at two wavelengths, r is a ratio of measured intensities at detectors, D is a distance between the emitter and detector, and A and B are lump constants for extinction coefficients, of Hb and HbO₂.

19. An apparatus for measuring brain oxygen saturation comprising:
a probe housing;
a near-infrared light emitter housed within the probe housing;
a first photodiode detector positioned a first distance from the emitter;
a second photodiode detector positioned at a second distance from the emitter, the second distance being longer than the first distance; and
a third photodiode detector positioned at a third distance from the emitter, the third distance being longer than the second distance.

20. The apparatus of claim 19, wherein the probe housing includes an apparatus for securing the housing to the head of a patient.
